(19) United States
(12) Patent Application Publication
(10) Pub. No.: US 2004/0177868 A1
(43) Pub. Date: Sep. 16, 2004

(54) Portable spray wash apparatus and method

(76) Inventor: Richard J. Kim, Great Falls, VA (US)

Correspondence Address: Richard J. Kim, 1004 Springvale Rd., Great Falls, VA, 22066, US

(21) Appl. No.: 10/384,900

(22) Filed: Mar. 11, 2003

(51) Int. Cl. B08B 3/02

(52) U.S. Cl. 134/36; 134/172; 134/198; 401/137; 401/268; 239/114; 239/123; 134/123

(57) ABSTRACT

A portable spray wash apparatus is self contained with a spray-wash head attached to an end of a wand having a grip handle at the other end. The grip handle is connected to one end of a flexible tube, the other end of the flexible tube being connected to a connection tap located at a lower portion of a fluid container. The apparatus achieves self-contained, light-weight portability for a convenient spray washing, regardless of the location, weather and/or other environmental constraints. It conserves water, and may be in-container mixed with a proper fluid mixture for environmentally friendly and socially responsible upkeep of vehicle appearance through all weather conditions.

### BACKGROUND OF INVENTION

Spray wash devices are known for vehicle washing. For example, U.S. Pat. No. 6,227,745 to Privett discloses a vehicle washing device comprising a hollow tube having a brush. Privett's vehicle washing device secures to a water hose. Thus, Privett's vehicle washing device serves as a spray wash wand requiring a separate source of water from an external hose connection.

Commercial vehicle laundry implements and their cloth elements are also known. For example, U.S. Pat. No. 6,035,482 to Belanger et al. discloses a rotatable vehicle laundry brush, and U.S. Pat. No. 6,279,190 to Belanger et al. discloses a contact type vehicle laundry element utilizing cloth-like surface contacting elements. However, they are intended for automated commercial facilities.

### SUMMARY OF INVENTION

The present invention provides for a shoulder-strapped and hand-gripped spray wash apparatus that is completely portable. The portable spray wash apparatus is self contained with a spray-wash head attached to a wand with a grip handle at the other end, the grip handle being connected by a flexible tube, the other end of the flexible tube being connected to a connection tap located at a lower portion of a fluid container. It achieves self-contained, light-weight portability for a relatively effortless and effective spray washing, regardless of the location, weather and/or other environmental constraints. It conserves water, and may be specially adapted with a proper wash fluid mixture for environmentally friendly and socially responsible upkeep of vehicle appearance through all weather conditions.

Its use and operation does not require an external hose connection, and does not require an electrical support. Instead, the spray-and-wash operation is gravity fed from a shoulder-strapped carrying position, and may be refilled from the carrying position through a refill opening on top of the fluid container. For in-door refilling, the refill may be from the likes of a kitchen sink, well protected from any outside weather conditions. The refill opening is normally twist sealed with a cap during storage or transport.

The gravity feed operation may be prepared by a partial twist-opening of the cap on top. The fluid flow may be actuated by hand actuation of a normally closed valve located at a holding position of the grip handle of the wand.

The spray-wash fluid container may be stored empty for filling at the time of use, or it may be stored pre-filled for ready use. When stored pre-filled, the refill cap may be twist closed. A spigot may be provided at the lower connection tap of the fluid container which connects to the flexible tube. The flexible tube may be stored disconnected, the spray-wash head being disassembled from the wand for compact storage and transportation.

The present invention does not require a pre-filling with a proprietary wash fluid. The strap-on fluid container is reusable and refillable with any fluid, including any combination of water, wash solvents, a soap solution, a wax or coating solution, a decontamination fluid, a bio-hazard sterilization solution; and a plant-food solution.

Thus, the portable spray wash apparatus is not limited to vehicle spray washing. A spray-wash head may be chosen or replaced based on the intended use, e.g., vehicle spray-washing; vehicle or equipment decontamination; personnel field hygiene and/or decontamination; outdoor pet grooming; window washing; or as a part of any field operation, including cooking, skin cleansing or first-aid cooling for heat-related injuries.

### BRIEF DESCRIPTION OF DRAWINGS

FIG. 1a is a perspective view of one example of a portable spray wash apparatus with a disassembled wand.

FIG. 1b is a perspective view of another example of a portable spray wash apparatus with a unitized wand of plastic extrusion.

FIG. 2a illustrates an example spray-brush head adaptable to the wand.

FIG. 2b illustrates an example spray-sponge head adaptable to the wand.

FIG. 2c illustrates an example spray-cloth head adaptable to the wand.

FIG. 2d illustrates an example spray-window wash head adaptable to the wand.

FIG. 2e illustrates an example spray head adaptable to the wand.

FIG. 3 illustrates an example connection tap with a spigot located at a lower portion of the fluid container in relation to a second end of a flexible tube securable with a flexible clamp.

FIG. 4 illustrates an example grip handle at the other end, the grip handle shown in relation to a first end of the flexible tube securable with another flexible clamp.

### DETAILED DESCRIPTION

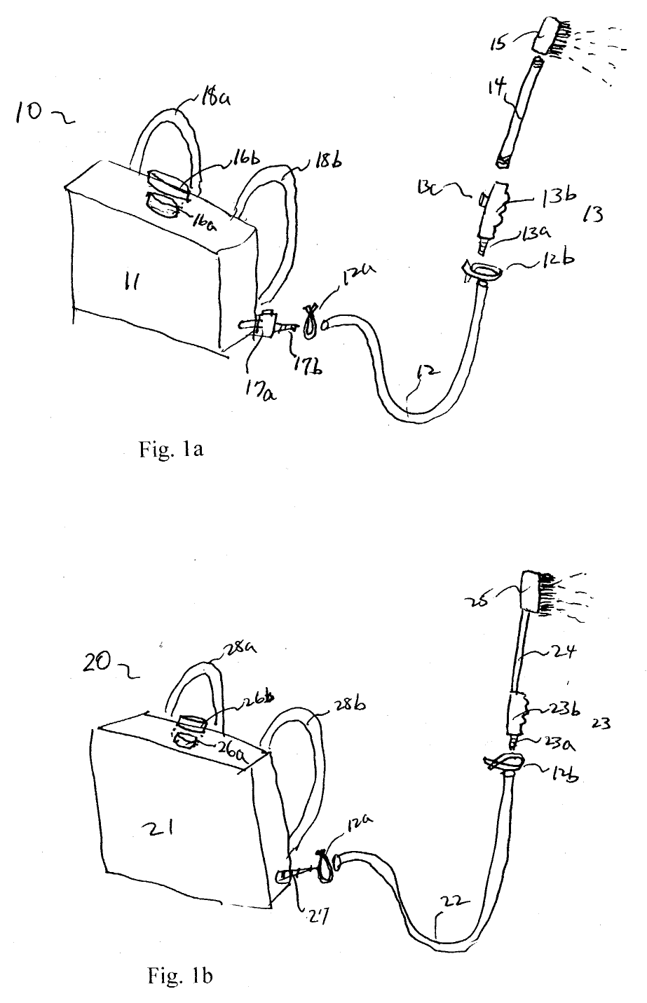

As exemplified in FIG. 1a, the preferred embodiment of the portable spray wash apparatus 10 is self contained with a spray-wash head 15 screw-recessed to mate with a first screw-grooved end of a wand 14. The wand 14 is preferably metallic in composition and screw-mated with a first end of a grip handle 13 at the other end of the wand 14, the grip handle 13 being connected at the other end 13a by a first end of a flexible tube 12, the second end of the flexible tube 12 being connected to a connection tap 17b located at a lower portion of a fluid container 11, which is preferably plastic extruded with a common recyclable High Density Polyethylene (HDPE). The grip handle 13 is preferably plastic injection molded, and may be surface rubberized for improved gripping and insulation.

The fluid container 11 has shoulder straps 18a and 18b for carrying. The spray-and-wash operation is gravity fed from a shoulder-strapped carrying position, and may be refilled from a shoulder-strapped carrying position through a refill opening 16a on top of the fluid container 11. However, for in-door refilling, the refill may be from the likes of a kitchen sink, well protected from any outside weather conditions. The refill opening is normally twist sealed with a cap during storage or transport.

The gravity feed operation may be prepared by a partial twist-opening of the cap 16b on top. Referring to FIG. 4, the fluid flow may be actuated by hand actuation of a normally closed valve 13c located at a holding position of the grip handle 13b. The valve 13c may be a spring-loaded, push-button fluid valve for convenient actuation from a holding position.

The spray-wash fluid container may be stored empty for filling at the time of use, or it may be stored pre-filled for ready use. When stored pre-filled, the refill cap may be twist closed. A spigot 17a may be provided at the lower connection tap 17b of the fluid container which connects to the flexible tube. The flexible tube 12 may be stored disconnected, the spray-wash head being disassembled from the wand for compact storage and transportation.

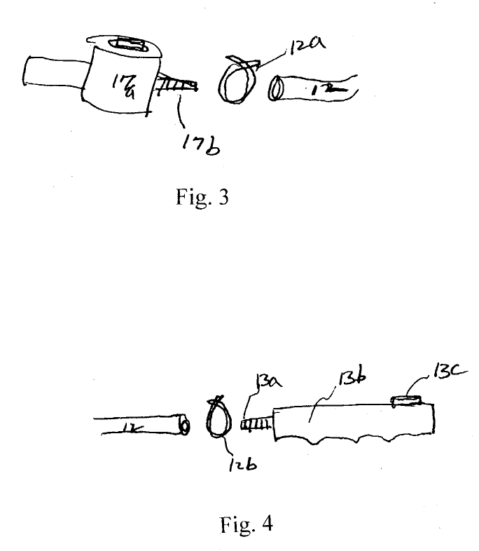

Referring to FIGS. 3 and 4, the first end of the flexible tube is clamped 12b to an end 13a of the grip handle 13 and the second end of the flexible tube 12 is clamped 12a to the connection tap 17b of the fluid container 11.

Another embodiment as shown in FIG. 1b unitizes the grip handle 23, wand 24, and the spray-wash head 25 as a plastic injection molding. For economy of production, the alternative embodiment is shown in FIG. 1b without a spigot. For such a variant, the connection tap 27 may be produced with its tap end sealed for nipping at the time of use.

The fluid container 11 or 21 is reusable and refillable with any fluid, including any combination of water, wash solvents, a soap solution, a soluble wax or coating solution, a decontamination fluid, a bio-hazard sterilization solution; and a plant-food solution. If the refillable wash fluid comprises a tap water, the temperature of the fluid may be quickly and conveniently controlled from the tap source.

A concentrated wash or coating solution may be formulated for in-container 11 or 21 dilution for effective, yet environmentally friendly, use with the portable spray wash apparatus. However, any such container solution may be achieved through measured in-container 11 or 21 dilution of a water-based solvent with a concentrated soluble composition.

Generally, the use of a spray wash apparatus 10 or 20 may comprise choosing and pouring a measured quantity of a soluble chemical into the top opening 15a or 26a of the fluid container 11 or 21, in-container dilution of the soluble chemical with water, strapping the portable spray wash apparatus 10 or 20 onto the shoulders, gravity-feed dispensing of the in-container dilution using the wand 14 or 24, and applying a spray-wash head 15 or 25 attached to the wand 14 or 24 to an application surface while simultaneously gravity-feed dispensing the in-container dilution to the same application surface.

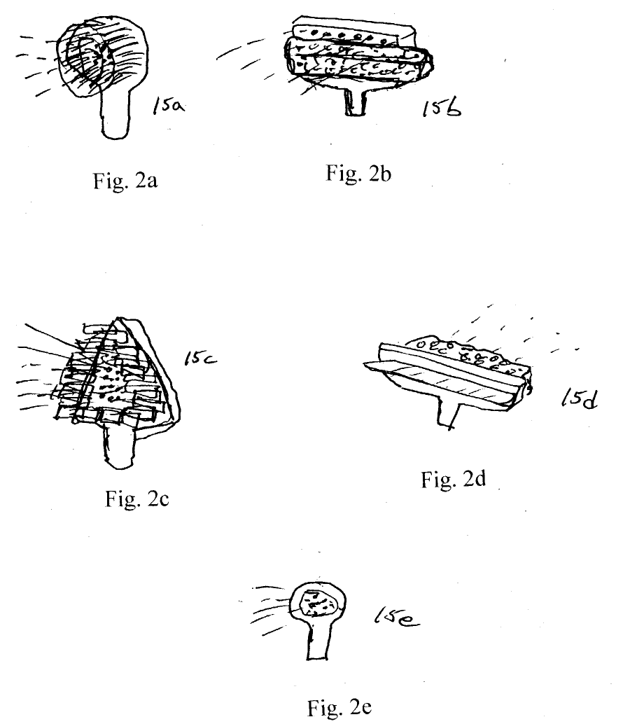

The use of a spray wash apparatus 10 or 20 of FIG. 1a or 1b is not limited to vehicle spray washing. Referring to FIGS. 2a-2e, the spray-wash head 15 or 25 may be any one of a spray-brush head 15a, a spray-cloth head 15b, a spray-sponge head 15c, a spray-window wash head 15d, and/or a spray head 15e of a variety of spray dispersion and/or flow rate. A spray-wash head 15 or 25 may be chosen or replaced based on the intended use, e.g., vehicle spray-washing; vehicle or equipment decontamination; personnel field hygiene and/or decontamination; outdoor pet grooming; window washing; or as a part of any field operation, including cooking, skin cleansing or first-aid cooling for heat-related injuries.

Therefore, the foregoing is considered as illustrative only of the principles of the invention. For example, various modifications, such as adhesion of a foam or insulating padding on a contact surface of the fluid container 11 and 2 for improved strap wear, all fall within the scope of the invention.

What is claimed is:

1. A portable spray wash apparatus having shoulder straps for carrying, comprising:
a wand having one end and another end;
a spray-wash head attached to the one end of the wand;
a grip handle having one end attached to the another end of the wand, another end of the grip handle being connected to one end of a flexible tube; and
a fluid container having a connection tap located at its lower portion connected to another end of the flexible tube, the fluid container having a top opening with a sealing cap.

2. The portable spray wash apparatus of claim 1, wherein the spray-wash head may be chosen from the group comprising a spray-brush head, a spray-cloth head, a spray-sponge head, a spray-squeegee head, and a spray head.

3. The portable spray wash apparatus of claim 1, wherein the grip handle, the wand, and the spray-wash head are formed from a plastic injection molding.

4. The portable spray wash apparatus of claim 1, wherein the wand is of a metallic composition.

5. The portable spray wash apparatus of claim 1, wherein a spigot is provided at the connection tap.

6. The portable spray wash apparatus of claim 1, wherein an actuating valve is provided at a holding position of the grip handle.

7. A method of use of a portable spray wash apparatus having shoulder straps for carrying, comprising:
pouring a measured quantity of a soluble chemical into a top opening of a fluid container;
in-container dilution of the soluble chemical with water;
strapping the portable spray wash apparatus onto the shoulders;
gravity-feed dispensing of the in-container dilution using a wand; and
applying a spray-wash head attached to the wand to an application surface while simultaneously gravity-feed dispensing the in-container dilution to the same application surface.

8. The method of use of a portable spray wash apparatus of claim 7, wherein the soluble chemical is chosen from the group comprising a carwash solution, a wax solution, a coating solution, a soap solution, a window wash solution, a decontamination chemical, a bio-hazard sterilization chemical, and a plant-food chemical.

9. The method of use of a portable spray wash apparatus of claim 7, wherein the water temperature is controlled from the source of water.
